(19) United States
(12) Patent Application Publication
(10) Pub. No.: US 2007/0185555 A1
(43) Pub. Date: Aug. 9, 2007

(54) MANUALLY ACTIVATABLE CHEMICAL COLD PACK

(76) Inventors: Myrna Partrich, Bloomfield Hills, MI (US); Rose Handleman, Bloomfield Hills, MI (US); Shelley Tauber, West Bloomfield, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/672,169

(22) Filed: Feb. 7, 2007

Related U.S. Application Data
(60) Provisional application No. 60/771,543, filed on Feb. 8, 2006.

(51) Int. Cl. A61F 7/00; F25D 3/08

(52) U.S. Cl. 607/114; 607/96; 62/530

(57) ABSTRACT

A cold pack comprising a handheld flexible plastic bladder filled with a smaller container of a first reactant, with a volume between the container and the bladder filled with a second reactant. The cold pack may be hand crushed to break the thin walled inner container, allowing the reactants to mix and generate an endothermic reaction cooling the outer walls of the bladder.

### CROSS-REFERENCE TO RELATED APPLICATIONS

This application claims priority of U.S. Provisional Patent Application Ser. No. 60/771,543 filed Feb. 8, 2006, which is incorporated herein by reference.

### FIELD OF THE INVENTION

This invention relates to a cold pack of the type having two fluid compounds in separated compartments, at least one of which may be broken with manual pressure to mix the compounds and generate an endothermic chemical reaction that cools the exterior of the pack, and more particularly to such a pack of such a size and configuration as to allow it to be crushed by a single hand and conveniently used in a variety of situations.

### BACKGROUND OF THE INVENTION

A variety of chemical cold packs exist which employ a pair of compounds in fluid form which are packaged in separate compartments of a single container. The container has flexible walls so that when it is crushed or kneaded at least one of the compartments breaks and mixes the two fluid compounds which react chemically with one another. The reaction is an endothermic one so that the exterior of the pack is lowered in temperature.

A variety of chemical compounds are used in these devices which are generally classified in class 62, subclass 004 of the United States Patent Office classification system.

### SUMMARY OF THE INVENTION

The preferred embodiment of the invention utilizes water as one reactant and a compound consisting of ammonium, nitrate sodium carboxyl, methyl cellulose and sodium chloride as the other reactant. Preferably, the water is retained in a soft plastic bladder which may be easily crushed by a single hand to release the water into the other chemical component. Both components are encased in a soft plastic waterproof case, preferably encased in a comfortable fabric such as cotton.

In a preferred embodiment of the invention the entire unit is generally palm sized and is generally disc shaped with a maximum diameter of about four inches. This allows the pack to be grasped in the palm of a hand and crushed by closing the hand into a fist so as to break at least one of the chemical bladders and intermix the chemical compounds, generating the endothermic reaction.

The pack of the present invention is intended to be carried in a woman's purse or the like so that it may be used in emergency situations which would benefit from the application of a cold pack. By way of example, the pack could be carried by women who are undergoing menopause and suffer from occasional hot flashes. In a social situation where other treatments might be embarrassing, the cold pack of the present invention could simply be removed from a purse and crushed in a single hand to provide cooling to the palm of the hand. Alternatively, the broken pack could be held against the user's neck or the like in an unobtrusive manner. The cold pack of the present invention could be used in other emergencies in which cold treatment is desired, such as bumps or bruises or the like.

### BRIEF DESCRIPTION OF TIE DRAWINGS

A preferred embodiment of the invention is illustrated in the accompanying drawings in which:

FIG. 1 is a top perspective view, from a side, of a cold pack forming a preferred embodiment of the invention;

FIG. 2 is a side view of the pack of FIG. 1;

FIG. 3 illustrates the cold pack of the present invention being supported in the palm of a user's hand before crushing; and

FIG. 4 illustrates the act of crushing the cold pack in a user's hand to generate the endothermic reaction that provides the cooling effect.

### DETAILED DESCRIPTION OF THE INVENTION

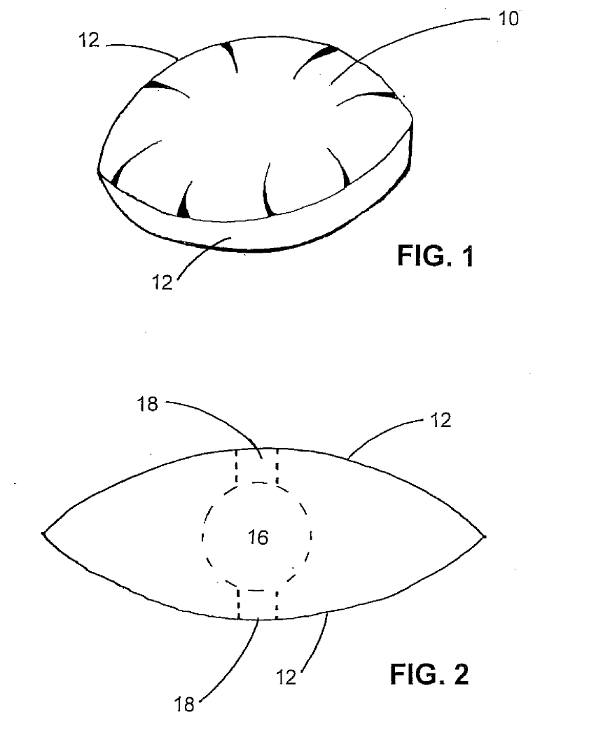

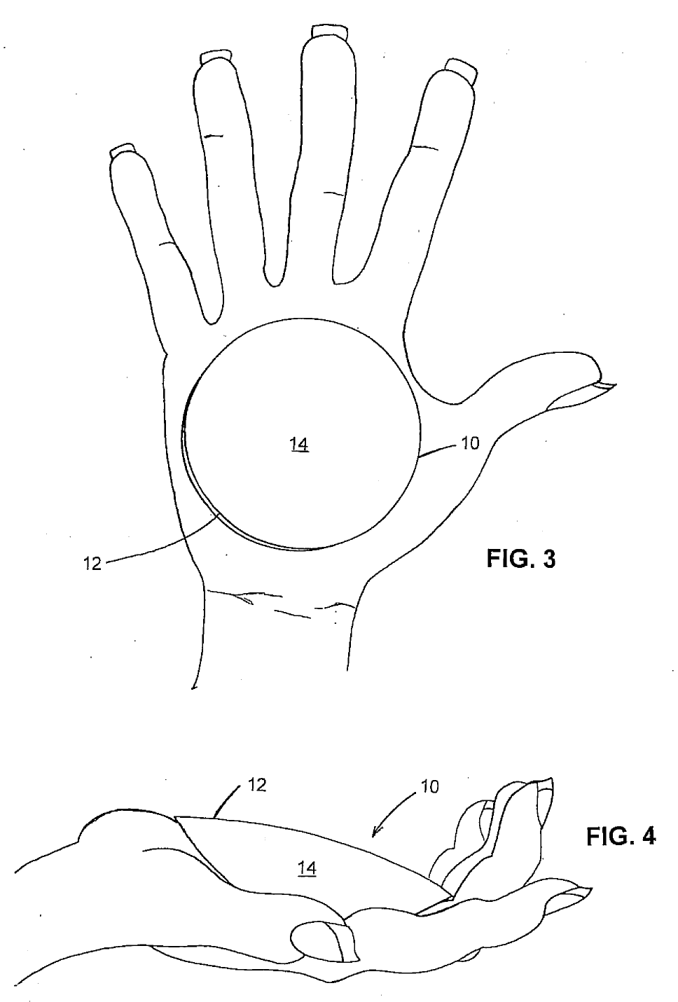

Referring to the drawings, the cold pack of the present invention, generally indicated at 10, is preferably disc shaped with a round edge 12 and tapered from a thickness of approximately ¼-½ inch at the center toward the edges 12 as shown in FIG. 2. As illustrated in FIG. 3, the dimensions of the outer container are preferably such that it fits within the user's hand. The outer skin of the container is preferably a flexible plastic sheet 14. Two sheets of fluid impervious flexible plastic such as polyethylene may be sealed at their circular edges to form a bladder shape. Within the bladder there is at least one thin flexible sheet plastic container 16 filled with one of the fluid reactants, preferably water, which is easily broken when the bladder is crushed in the hand in the manner illustrated in FIG. 4. The container 16 is stabilized within the bladder by sheet plastic connector 18. This releases the first component, such as water, into contact with the reacting chemical, which is in fluid form and fills the volume within the cold pack bladder on the exterior of the sheet plastic container. In other embodiments of the invention both the bladder and the interior body of the pack could contain different reacting chemicals in fluid form, in any of the types disclosed in class 62/004. The preferred embodiment employs a solution of ammonium chloride.

Having thus described our invention, we claim:

1. A cold pack comprising:
two circular sheets of fluid impervious plastic film sealed at their edges to form a disc-shaped bladder enclosing a first volume;
a thin plastic sheet container filled with a first fluid reactant, having a second, smaller volume than the first volume; and
a second fluid reactant, which produces an endothermic reaction when mixed with the first reactant disposed in the bladder on the exterior of the sheet container;
whereby, the bladder may be compressed to rupture the thin plastic sheet container to allow the first and second reactants to intermix generating an endothermic reaction cooling the surface of the bladder.

2. The cold pack of claim 1 in which the bladder has a thickness of about ½ inch at the center.

3. The cold pack of claim 2 in which the first reactant is water and the second reactant is ammonium chloride.
